(19) United States
(12) Patent Application Publication
(10) Pub. No.: US 2022/0230740 A1
(43) Pub. Date: Jul. 21, 2022

(54) METHOD AND COMPUTER PROGRAM TO DETERMINE USER'S MENTAL STATE BY USING USER'S BEHAVIOR DATA OR INPUT DATA

(71) Applicant: RFCAMP LTD., Gyonggi-do (KR)

(72) Inventors: Jae Hyung RYU, Gyeonggi-do (KR); Kwon Soo KIM, Seoul (KR); Ji Youn KIM, Gyeonggi-do (KR)

(73) Assignee: RFCAMP LTD., Gyonggi-do (KR)

(21) Appl. No.: 17/567,853

(22) Filed: Jan. 3, 2022

(30) Foreign Application Priority Data
Jan. 21, 2021 (KR) 10-2021-0008789

(51) Int. Cl. G16H 40/63; G06F 16/901

(52) U.S. Cl. CPC G16H 40/63; G06F 16/9017

(57) ABSTRACT

A mentality determination service is a service that provides the mental state of a user who is a psychology subject by connecting the user who is a psychology subject to a user who is a psychological analysis counselor, and may include a service that automatically calculates the mental state, based on data created by the psychology subject. The mentality determination service may automatically calculate the mental state of the user by using a relationship equation between input factors created according to a statistical method and a mental state corresponding to an output.

### CROSS-REFERENCE TO RELATED APPLICATION

This application is based on and claims priority under 35 U.S.C. § 119 to Korean Patent Application No. 10-2021-0008789, filed on Jan. 21, 2021, in the Korean Intellectual Property Office, the disclosure of which is incorporated by reference herein in its entirety.

### BACKGROUND

### 1. Field

One or more embodiments relate to a method and computer program to determine a mental state of a user by using behavior data or input data of the user.

### 2. Description of the Related Art

In psychology, human psychology is defined based on five major factors of personality traits: O (Openness), C (Conscientiousness), E (Extraversion), A (Agreeableness), and N (Neuroticism).

Openness is related to imagination, aesthetics, emotion, and ideas; conscientiousness is related to order, responsibility, pursuit of achievement, moderation, and reflection; extraversion is related to sociability, confidence, stimulation seeking, and positive emotions; agreeableness is related to honesty, obedience, humility, and tenderness; and neuroticism is related to anxiety, hostility, depression, self-consciousness, impulsivity, and stress sensitivity. The metal state of a person may be defined as O, C, E, A, or N by a psychoanalytic counselor.

### SUMMARY

One or more embodiments include a method and computer program to determine a user's mental state by using user's behavior data or input data.

Additional aspects will be set forth in part in the description which follows and, in part, will be apparent from the description, or may be learned by practice of the presented embodiments of the disclosure.

According to one or more embodiments, a method, performed by an electronic device, of determining a mental state of a user by using behavior data or input data of the user, includes executing a mental state determiner; obtaining first setting data, first shared data, and first search data, based on behavior data or input data according to a behavior of the user who is using the mental state determiner; obtaining first mentality data created by a first user; calculating a mental state value by applying the first mentality data to a mentality-state table; calculating a first mentality value by applying the first setting data to a mentality-setting table; calculating a second mentality value by applying the first sharing data to a mentality-sharing table; calculating a third mentality value by applying the first search data to a mentality-search table; and calculating a final mental state value by correcting the mental state value to the first through third mentality values.

The first mentality data may include at least one of an emotional word set input by the user and drawing data input by the user.

The first setting data may include at least one of a subscription path, an subscription app, subscription date and time, a subscription region, a charging time point, profile registration information, an access device, access date and time, an access duration, an access frequency, an access location, an access proportion, an access environment, and environment setting information, which are related to a subscription behavior of the user.

The first sharing data may include whether mentality data created by the user is shared, sharing information, upload percentage information, sharing percentage information to other platforms, direct transmission percentage information of pieces of mentality data, data of preference actions for mentality data of other users, and data of a preference action for the user.

The first search data may include at least one of inquiry/reading information for a psychological analysis counselor, inquiry/reading information for a psychological subject, and inquiry/reading information for the mentality data.

The mental state value may include a value corresponding to openness, conscientiousness, extraversion, agreeableness, neuroticism, and energy related to the mental state of the user.

The first through third mentality values may include a value corresponding to openness, conscientiousness, extraversion, agreeableness, neuroticism, and energy related to the mental state of the user.

At least one of the mentality-state table, the mentality-setting table, the mentality-sharing table, and the mentality-search table may represent a correlation between at least one of one or more pieces of mentality data, setting data, shared data, and search data input by the user and a mental state corresponding to the at least one of the one or more pieces of mentality data, setting data, shared data, and search data.

At least one of the mentality-state table, the mentality-setting table, the mentality-sharing table, and the mentality-search table may be created by being trained with training data that uses at least one of pre-created one or more pieces of mentality data, setting data, shared data, and search data as an input and uses pre-generated one or more mental states as an output.

A computer program according to an embodiment of the present disclosure may be stored in a medium to execute one of methods according to embodiments of the present disclosure by using a computer.

One or more embodiments include another method for implementing the present disclosure, another system, and a computer-readable recording medium for recording a computer program for executing the other method.

These and/or other aspects will become apparent and more readily appreciated from the following description of the embodiments, taken in conjunction with the accompanying drawings.

### BRIEF DESCRIPTION OF THE DRAWINGS

The above and other aspects, features, and advantages of certain embodiments of the inventive concept will be more apparent from the following description taken in conjunction with the accompanying drawings, in which:

FIG. 1 is a block diagram of an electronic device according to embodiments of the present disclosure;

FIG. 2 is a block diagram of a mental state determiner according to embodiments of the present disclosure;

FIG. 3 is a diagram of a mentality determination system according to embodiments of the present disclosure;

FIG. 4 is a flowchart of a mentality determination method according to embodiments of the present disclosure;

FIG. 5 is a road map of pieces of data obtained by the mental state determiner to determine the mental state of a user;

FIG. 6A is first mentality-factor table representing a correlation between data and a mental state value used in embodiments of the present disclosure; FIG. 6B is second mentality-factor table representing a correlation between data and a mental state value used in embodiments of the present disclosure; FIG. 6C is third mentality-factor table representing a correlation between data and a mental state value used in embodiments of the present disclosure;

FIG. 7 illustrates a mentality-drawing table used in embodiments of the present disclosure;

FIG. 8 illustrates a mentality-emotional word table used in embodiments of the present disclosure;

FIG. 9 illustrates a mentality-setting table used in embodiments of the present disclosure;

FIG. 10A illustrates a mentality-setting table used in embodiments of the present disclosure; FIG. 10B illustrates a mentality-search table used in embodiments of the present disclosure;

FIG. 11A illustrates a table of description and criterion among a mentality-factor table used in embodiments of the present disclosure; FIG. 11B illustrates a table of magnitude values of each factor among a mentality-factor table; and FIG. 11C illustrates a table of mental state values for factors among a mentality-factor table.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “one or more of,” when preceding a list of elements, modify the entire list of elements and do not modify the individual elements of the list.

Hereinafter, the present disclosure will be described more fully with reference to the accompanying drawings, in which exemplary embodiments of the present disclosure are shown.

As the present disclosure allows for various changes and numerous embodiments, particular embodiments will be illustrated in the drawings and described in detail in the written description. The attached drawings for illustrating exemplary embodiments are referred to in order to gain a sufficient understanding of the effects and features thereof, and methods for accomplishing the effects and features thereof. However, this is not intended to limit the present disclosure to particular modes of practice, and it is to be appreciated that all changes, equivalents, and substitutes that do not depart from the spirit and technical scope are encompassed in the present disclosure.

Hereinafter, the present disclosure will be described in detail by explaining exemplary embodiments of the present disclosure with reference to the attached drawings. Like reference numerals in the drawings denote like components, and thus their description will be omitted.

It will be understood that the terms used herein, such as “training,” “learning,” etc. are not intended to refer to mental actions such as human educational activities but refer to performing machine learning through computing according to procedures.

It will be understood that although the terms “first”, “second”, etc. may be used herein to describe various components, these components should not be limited by these terms. These components are only used to distinguish one component from another.

As used herein, the singular forms “a”, “an”, and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

Sizes of components in the drawings may be exaggerated for convenience of explanation. In other words, since sizes and thicknesses of components in the drawings are arbitrarily illustrated for convenience of explanation, the following embodiments are not limited thereto.

When a certain embodiment may be implemented differently, a specific process order may be performed differently from the described order. For example, two consecutively described processes may be performed substantially at the same time or performed in an order opposite to the described order.

A mentality determination service is a service that provides the mental state of a user who is a psychology subject by connecting the user who is a psychology subject to a user who is a psychological analysis counselor, and may include a service that automatically calculates the mental state, based on data created by the psychology subject. The mentality determination service may automatically calculate the mental state of the user by using a relationship equation between input factors created according to a statistical method and a mental state corresponding to an output. In addition, the mentality determination service may use a model created by a machine learning algorithm using an artificial neural network. The mentality determination service may extract the types of input factors used to calculate the mental state of the user suggested by a devised learning model, and may input the extracted input factors to the learning model to calculate the mental state corresponding to an output. The learning model may output the mental state as an output by applying weight values applied to the input factors.

The psychology subject may use the mental determination service by installing, in an electronic device, a mental state determiner implemented to provide the mental determination service. In addition to a function of determining the mental state of a user, the mental state determiner may provide a sharing platform function for sharing input data with other users, a posting function for posting the data input by the user, and the like.

The mentality determination service is performed by a program installed in an electronic device, and may be performed in conjunction with an external management server.

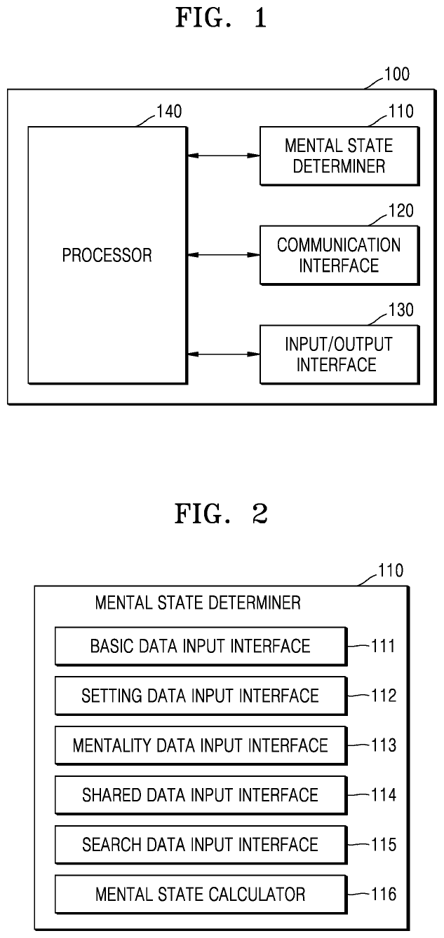

FIG. 1 is a block diagram of an electronic device 100 according to embodiments of the present disclosure.

The electronic device 100 may include a mental state determiner 110, a communication interface 120, an input/output interface 130, and a processor 140.

The mental state determiner 110 may be a set of one or more instructions. The mental state determiner 110 may be implemented as a computer-readable medium. The mental state determiner 110 may be a random access memory (RAM), a read only memory (ROM), and a permanent mass storage device such as a disk drive. The mental state determiner 110 may be a computer-readable recording medium such as a floppy drive, a disk, a tape, a digital video disc/compact disc read-only memory (DVD/CD-ROM) drive, or a memory card.

The communicator 120 may provide a function for communicating with an external device via a network. For example, a request generated by the processor 140 of the electronic device 100 according to a program code stored in a recording device such as the mental state determiner 110 may be transmitted to an electronic device 300, a database 200, or a management serve 400 via the network under the control by the communication interface 120. For example, a control signal or command received through the communication interface 120 may be transmitted to the processor 140, a storage medium, or the mental state determiner 110, and a received video image may be stored in the storage medium or the mental state determiner 110.

The input/output interface 130 may display a screen image providing information or receive an input from a user. For example, the input/output interface 130 may include an operation panel that receives a user input, and a display panel that displays a screen image.

In detail, the input interface may include devices capable of receiving various types of user inputs, such as a keyboard, a physical button, a touch screen, a camera, or a microphone. The output interface may include a display panel or a speaker. However, embodiments of the present disclosure are not limited thereto, and the input/output interface 130 may include a structure that supports various inputs and outputs.

The processor 140 may be implemented as one or more processors, and may be configured to process commands of a computer program by performing basic arithmetic, logic, and I/O operations. The commands may be provided to the processor 140 by the storage medium or the communication interface 120. For example, the processor 140 may be configured to execute a received command according to a program code stored in the recording device such as the mental state determiner 110 or a storage medium.

The electronic device 100 may further include a computer-readable recording medium, such as a RAM and a ROM, and a permanent mass storage device such as a disk drive.

FIG. 2 is a block diagram of the mental state determiner 110 according to embodiments of the present disclosure.

The mental state determiner 110 may include a basic data input interface 111, a setting data input interface 112, a mentality data input interface 113, a shared data input interface 114, a search data input interface 115, and a mental state calculator 116.

The mental state determiner 110 performs a function of determining a mental state of a user, a service for sharing mentality data of the user, a function of storing or sharing a mental state input by a psychological analysis counselor, a function of sharing the user's mentality data, user's mental state data, etc. with other users, and a function of searching for data of the other users. A logic relating to these functions may be included in the mental state determiner 110, or may be processed with data received from the management server 400 outside the electronic device 100.

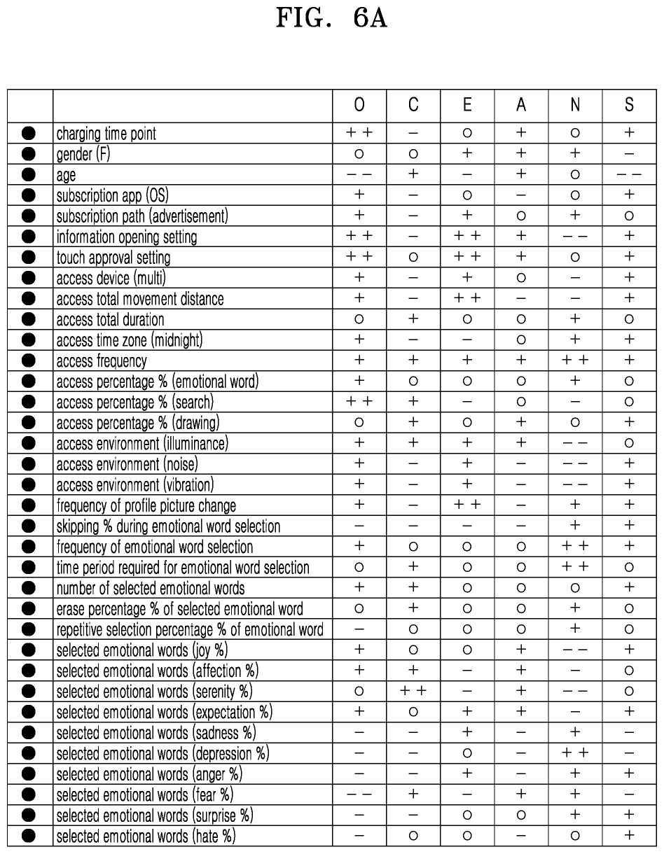

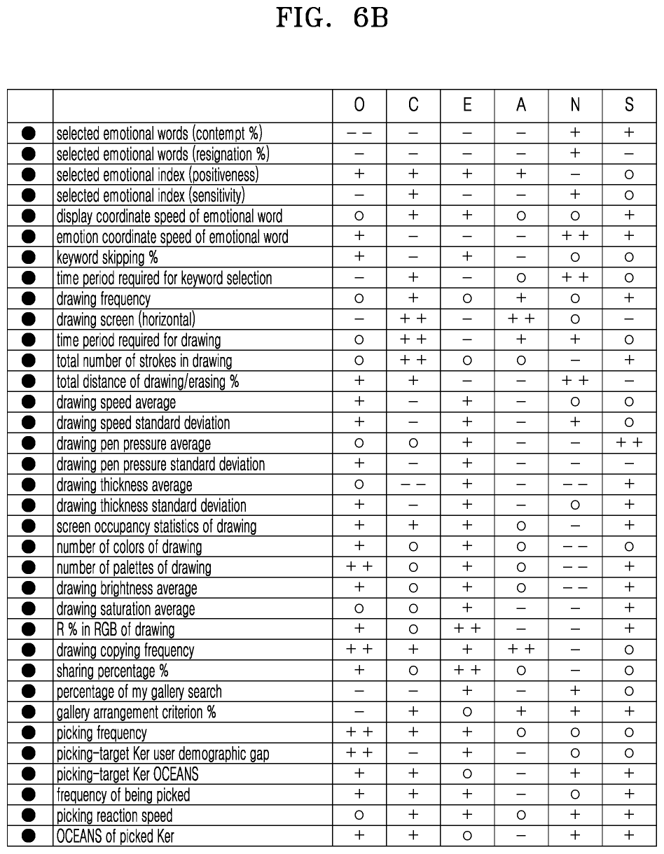

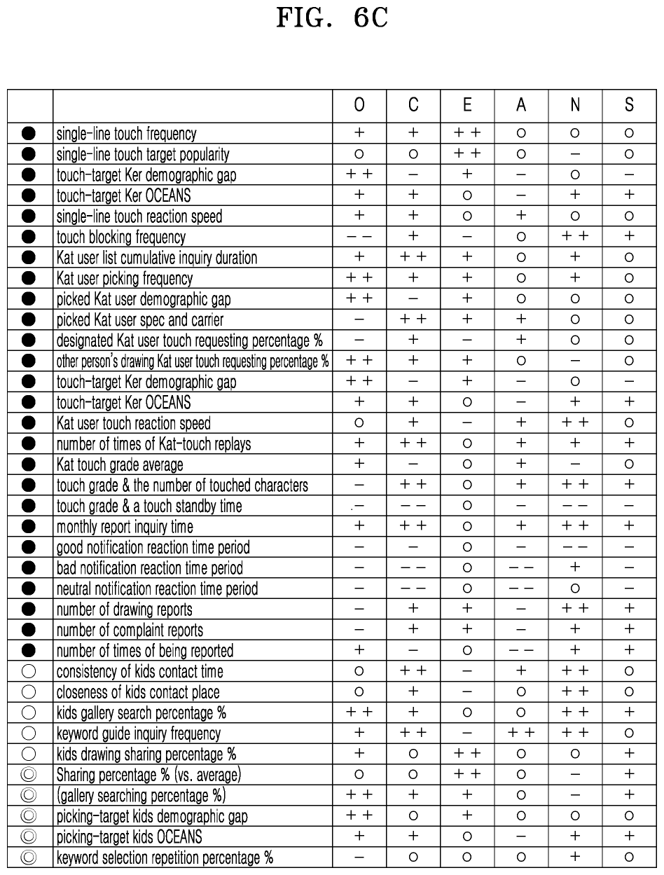

The mental state determiner 110 may estimate and calculate a mental state value of the user, based on the user's behavior data or input data obtained while providing a service. The behavior data is data related to a user's behavior of using the electronic device 100, and may include screen activation-related information about the electronic device 100 (time point, number of times, etc.), execution related data of other applications (frequency of execution, execution frequency cycle, etc.), the number of applications executed in the background, and whether applications executed in the background are activated or not. However, embodiments of the present disclosure are not limited thereto, and the behavior data may include various data. The behavior data may include pieces of data of FIGS. 6A, 6B and 6C.

The input data is data related to values input by the user to the electronic device 100, and may include data of setting values for other applications, data of basic setting values of the electronic device 100, and the like. However, embodiments of the present disclosure are not limited thereto, and the input data may include various data. The input data may include the pieces of data of FIGS. 6A, 6B and 6C.

In this case, the mental state or the mental value may be set for each type such as openness, conscientiousness, extraversion, agreeableness, neuroticism, and energy. However, embodiments of the present disclosure are not limited thereto, and the mental state may be defined as other various types. The mental state or the mental value of the user may be determined based on basic data about the user, setting data related to the user's behavior, shared data, search data, and input data input by the user.

The basic data input interface 111 receives basic data about the user. The basic data is data registered when the user registers in the mentality determination service, and may include, for example, the user's ID information, gender, age, nationality, hair color, eye color, living area, and the like.

The basic data may be transmitted to and stored in the database 200. The basic data input interface 111 may input basic data received from the database 200.

The factors of the basic data may be classified according to a predetermined rule. The factors of the basic data may be calculated according to a predetermined rule. For example, basic data related to an ID may include the number of types of codes included in the ID and/or password, the total number of characters in the ID and/or password, whether the ID and/or password include numbers, and whether the ID and/or password include special characters. The classified factors may be values defined in correspondence with pieces of corresponding information.

All or some from among pieces of basic data may be extracted in consideration of the degree of relevance to the determination of the mental state in order to calculate the mental state. For example, when the degree to which whether the ID and/or password include numbers is related to the determination of the mental state is lower than a preset basic determination degree, whether the ID and/or password include numbers may be excluded from an extraction target for the determination of the mental state.

The setting data input interface 112 receives setting data associated with a behavior of the user. The setting data may include a subscription path, a subscription app, subscription date and time, a subscription region, a charging time point, profile registration information, an access device, access date and time, an access time period (time period in a day, a week, or a month), an access frequency (frequency in a day, a week, or a month), an access location (street name, cumulative distance traveled between access locations, etc.), an access weight (emotional word selection percentage information, search percentage information, drawing percentage information, touch percentage information, alarm confirmation percentage information, a monthly report, etc.), an access environment, environment setting information, and the like, which are related to a user's subscription behavior.

The environment setting information in the setting data may include whether location information is provided, whether a profile is disclosed, whether environmental information (illuminance, noise, vibration, etc.) is provided, whether a single-line touch is approved, whether a login approval of other devices is approved, whether a selected emotional word is disclosed, whether a selected keyword is disclosed, whether a mental state is disclosed, whether an emotional index is disclosed, whether a notification is set, whether a sound is set, screen settings (full screen, partial screen, etc.), the frequency of changes in setting information, and the like. However, embodiments of the present disclosure are not limited thereto, and the environment setting information in the setting data may include various setting values of the mental state determiner 110.

The subscription path in the setting data may include information about the path along which the user has subscribed to a corresponding mentality determination service. For example, the subscription path may be set to be, but is not limited to, at least one of inflow through an advertisement on a video sharing platform, inflow through advertisement on an SNS platform, inflow through a link after portal search, inflow through shared (posted, put up) content, and inflow through a direct message.

The profile registration information in the setting data may include, but is not limited to, profile registration date and time, a profile registration location, a profile change time point, an emotional word set included in a profile, a keyword set included in the profile, attribute information of the profile, whether a mental state is disclosed, and whether an emotional index is disclosed.

All or some from among pieces of setting data may be extracted in consideration of the degree of relevance to the determination of the mental state. For example, first setting data of which the degree of relevance to the determination of the mental state is less than or equal to a preset basic determination degree may not be extracted and may be removed.

The mentality data input interface 113 receives mentality data from the user. The mentality data is input data related to direct determination of the mental state by the user, and may include, but is not limited to, an emotional word set selected and input by the user, drawing data created by the user, and the like, and may further include facial expression data, voice data, and the like. The mentality data may also include selection date and time, a selection location, a selection frequency, skip percentage information, a time period required for selection, the number of selected emotional words, the type of selected emotional word, a total required time period, time information until a first word is selected, time information until a last word is selected and then the selection is terminated, the number of selected and deleted emotional words, percentage information of deleted emotional words, percentage information of repeatedly selected emotional words, a dominant emotion (one of joy, affection, serenity, expectation, sadness, depression, anger, fear, surprise, hate, contempt, and resignation) set by an emotional word, emotional indexes (positiveness, sensitivity, etc.), display coordinate values of selected emotional words, emotion coordinate values of the selected emotional words, and correlation coefficients between the display coordinate values and the emotion coordinate values. The emotional coordinate value may be a combination of one or more values, such as a positive level value of an emotional word, a stimulus level value thereof, and the like. A positive level value and a stimulus level value of each emotional word may be set by an administrator or the like.

The mentality data may also include drawing date and time, a drawing place, drawing environment information, drawing frequency information, drawing screen settings (horizontal or vertical screen), a drawing duration, drawing attributes, an emotional index, a mental state value, drawing attribute information, and the like, which are related to input of drawing data.

The drawing attribute information may include the number of strokes within drawing data created by the user, the total number of strokes, a distance, a total distance, an erase distance, an erase percentage, a pause duration, pause percentage information, a speed, an average speed, a standard deviation of a speed, a pen pressure value, an average value of a pen pressure, a standard deviation of the pen pressure, a thickness value, an average value of thicknesses, a standard deviation of the thicknesses, a screen occupancy level value, an occupancy level value in a full screen, an occupancy level value for each split region in a 9-split screen, a color value, the total number of colors used, the total number of palettes used, percentage information of first through third color values (e.g., R, G, and B) among colors, an average of used brightness values, a standard deviation of used brightness values, an average of used saturation values, a standard deviation of saturation values, an average of used primary colors, a standard deviation of primary colors, a correlation coefficient value between the factors of at least two of the calculated values (distance-speed, distance-pen pressure, speed-pen pressure, thickness-pen pressure, brightness-pen pressure, saturation-pen pressure, a percentage-pen pressure of red among R, G, and B), and the like.

The mentality data refers to data input in relation to the mental state, and may further include a keyword selected and input by the user. The keyword may include factors such as reading information (reading year, reading time, etc.) of a keyword guide, skip percentage information when a keyword is selected, a time period required for selection, and subject type information during selection. The subject type information during selection may include a family, an emotion, a building, a game, the past, an animal, a cartoon, the future, a person, a situation, coloring, belongings, a plant, an appearance, a food, an event, a place, a task, the present, an environment, etc.

The shared data input interface 114 may input shared data associated with a sharing action of the user. The shared data input interface 114 may receive, from the database 200, shared data associated with a sharing action conducted by a first user.

The shared data may include whether mentality data created by the user is shared, the degree of sharing (public to all people, private, public to friends, etc.), upload percentage information from pieces of mentality data, sharing percentage information to other platforms, directly-transmitted percentage information of the pieces of mentality data, data of preference actions for mentality data of other users (number of times, ranking information of preference actions, attribute information of preferred data, user information of the preference actions, demographic information of user information, etc.), data of a preference action for the user (reaction speed of the preference action (time from an upload time point to an input of the preference action), attribute information of preferred mentality data, attribute information of the user, etc.), the frequency of single line touch information, popularity ranking of a single-line touch, attribute information of mentality data of the single-line touch, demographic information of a user of the mentality data of the single-line touch, data about reception of the single-line touch (reaction time until the single line touch is received), information of a user who touched one line, an emotional index of the user, a mental state value of the user, the frequency of a blocking action, user information of the blocking action, and the like.

The single-line touch is content input by one or more users with respect to the shared data, and may be in various formats such as text, an image, and a moving picture. The single-line touch may include analysis comments on all or some of the mentality data.

The search data input interface 115 may input search data associated with a search action of the user. The search data may include inquiry/reading information for a psychological analysis counselor, inquiry/reading information for a psychological subject, inquiry/reading information for the mentality data, and the like. The search data may be created by each user terminal and transmitted to and managed by the management server 400.

The mental state calculator 116 may create tables representing the relationship between the user's mental state and all or some of the basic data, the setting data, the mentality data, the shared data, and the search data. For example, the mental state calculator 116 may create a first mental state and all or some of basic data, setting data, mentality data, shared data, and search data obtained by one or more users in the first mental state in a mentality-factor table, thereby determining a mental state for a first factor obtained in real time. The mentality-factor table may be created for one or more mental states output by the mental state determiner 110. The mentality-factor table may include a mentality-basic table, a mentality-emotional word table, a mentality-drawing table, a mentality-setting table, a mentality-sharing table, a mentality-search table, and the like. The mentality-factor table may be defined according to the age of a psychological subject. For example, a table for users over 14 years of age and a table for users under 14 years of age may be different.

The mentality-factor table may be a two-dimensional table of a mental state or applied weight value and the name of a factor, and values in the mentality-factor table may be a correlation between a factor and a mental state, a relationship index therebetween, and the like. The mental state may be one of one or more types included in the mental state.

According to another embodiment, the mentality-factor table may be converted into a mentality-factor relationship equation in which all or some of basic data, setting data, mentality data, shared data, and search data are used as input variables and a mental state is used as an output variable. The mentality-factor relationship equation (term is ok?) may include a mentality-basic relationship equation, a mentality-emotional word relationship equation, a mentality-drawing relationship equation, a mentality-setting relationship equation, a mentality-sharing relationship equation, a mentality-search relationship equation, and the like. The mentality-factor relationship equation may be defined according to the age of a psychological subject. For example, a relationship equation for users over 14 years of age and a relationship equation for users under 14 years of age may be different.

The mentality-basic relationship equation uses the entirety or a portion of the basic data as an input variable and a mental state as an output variable, and may be obtained using data that links the basic data of users to the mental states of users, respectively.

The mentality-emotional word relationship equation uses the entirety or a portion of the emotional word data as an input variable and a mental state as an output variable, and may be obtained by linking data about emotional words selected and input by the users to the mental states of the users, respectively.

The mentality-drawing relationship equation uses the entirety or a portion of the drawing data as an input variable and a mental state as an output variable, and may be obtained by linking drawing data generated by the users to the mental states of the users, respectively.

The mentality-sharing relationship equation uses the entirety or a portion of the shared data as an input variable and a mental state as an output variable, and may be obtained by linking shared data associated with sharing actions of the users to the mental states of the users, respectively. The mentality-search relationship equation and the mentality-setting relationship equation may be created using a similar method to the above-described method.

The mentality-basic relationship equation, the mentality-emotional word relationship equation, the mentality-drawing relationship equation, the mentality-setting relationship equation, the mentality-sharing relationship equation, and the mentality-search relationship equation may be trained using basic data, emotional word data, drawing data, setting data, shared data, and search data obtained by mental state determiners provided in one or more electronic devices connected via a network and the mental states of the users. The management server 400 may create pieces of training data in which all or some of the basic data, the emotional word data, the drawing data, the setting data, the shared data, and the search data correspond to the mental states of the users, and may input the pieces of training data to an artificial neural network to thereby create at least one of the mentality-basic relationship equation, the mentality-emotion word relationship equation, the mentality-drawing relationship equation, the mentality-setting relationship equation, the mentality-sharing relationship equation, and the mentality-search relationship equation. This training method may be, but is not limited to, machine learning, reinforcement learning, unsupervised learning, or the like, and various methodologies may be used.

In this case, the mental state may be set for each type such as openness, conscientiousness, extraversion, agreeableness, neuroticism, and energy for experiences. However, embodiments of the present disclosure are not limited thereto, and the mental state may be defined as other various types. The energy refers to the degree of energy that the user has, and may be classified into and set as internal energy or external energy. The mental state may be set by an input by a psychological analysis counselor, and, after inputs by the psychological analysis counselor are accumulated and thus a table and/or relationship equation is created, the table and/or relationship equation may be used. The table and/or relationship equation may be created by various statistical programs, machine learning methods, reinforcement learning methods, and the like.

In this case, mental states used to create the table and/or relationship equation may be obtained from the terminals of one or more psychological analysis counselors, but embodiments of the present disclosure are not limited thereto. The mental states used to create the table and/or relationship equation may be obtained in various ways. A mentality-factor table or a mentality-factor relationship equation may be calculated based on mental states obtained in various ways.

A process of requesting the terminals of one or more psychological analysis counselors for mental states may be performed by the mental state determiner 110 or may be performed by the management server 400 in response to a request signal from the mental state determiner 110. For example, first emotional word data or first drawing data, which is mentality data of a first user, may be transmitted to a terminal of a psychological analysis counselor, and a first mental state corresponding to the first emotional word data or the first drawing data may be received from the terminal of the psychological analysis counselor. The first emotional word data may be based on a selection input by a user. The first drawing data may be based on not only a drawing input by the user and a touch input by the user but also color selection of a drawing, thickness selection of the drawing, and the like, which are pieces of detailed information about the drawing input and/or the touch input.

When a first mental state corresponding to the mentality data of the first user is received or calculated, the mental state calculator 116 may link the first mental state with one or more data (factor and attribute value) included in basic data, setting data, shared data, and search data of the first user and store a result of the linkage. Through this process, a mentality-factor table in which the first mental state corresponding to the mentality data of the first user is linked with data other than the mentality data of the first user may be created, and a mentality-factor relationship equation representing a correspondence between the data and the first mental state may be created based on the mentality-factor table.

According to another embodiment, an input by a psychological analysis counselor may be actually performed through an online consultation or a face to face consultation with a second user. An online conversation between the second user and the psychological analysis counselor may be performed using an online consultation program provided by the mental state determiner 110, and the psychological analysis counselor may input a second mental state of the second user to the terminal of the psychological analysis counselor, based on the online conversation and an action, an input, and the like of the second user, which occur in the online conversation, and may also input a single-line touch.

After the second mental state is received or calculated, the mental state calculator 116 may create a table in which the second mental state is linked with one or more data (factor and attribute value) included in basic data, setting data, shared data, and search data of the second user and a result of the linage is stored. This table may be converted into a pre-determined structure, and the pre-determined structure may be transmitted to and stored in the management server 400. The management server 400 may create a mentality-factor relationship equation representing a correspondence between a mental state and pieces of data by using a mentality-factor table in which the second mental state corresponding to the mentality data of the second user is linked with data other than the mentality data of the second user.

The mentality-factor table or the mentality-factor relationship equation may be generated using a determined logic. When inputs by psychological analysis counselors are accumulated and an accumulated capacity (dimension or size) exceeds a preset reference capacity, the mental state determiner 110 and the management server 400 connected to the mental state determiner 110 via a network may generate the mentality-factor table and the mentality-factor relationship equation by executing an algorithm for generating a table and/or a relationship equation. Based on the generated mentality-factor table and the generated mentality-factor relationship equation, the mental state determiner 110 may calculate the mental state of the user.

The logic for generating the mentality-factor table or the mentality-factor relationship equation may be created as follows. The entirety or a portion of the logic for generating the mentality-factor table or the mentality-factor relationship equation may be implemented in the mental state determiner 110 of an electronic device 100 or in the management server 400 connected to the mental state determiner 110 via a network. When setting data, shared data, and search data related to an action of the user other than emotional words input by the user and drawing data are collected to be equal to or greater than a preset basic cumulative capacity value, the mental state determiner 110 may determine the mental state of the user, based on at least one of the setting data, the shared data, and the search data of the user. Mental states of the user may be repeatedly determined at a reference period in which the setting data, the shared data, and the search data equal to or greater than the preset basic cumulative capacity value are collected. The basic cumulative capacity may be determined based on the capacity of data used (or having been used) to create each of the mentality-setting relationship equation, the mentality-sharing relationship equation, or the mentality-search relationship equation. When the mentality-setting relationship equation, the mentality-sharing relationship equation, or the mentality-search relationship equation is created from setting data, shared data, or search data of 100 megabytes, respectively, setting data, shared data, or search data of 10 megabytes, which is a certain portion of 100 megabytes, for example, 10%, may be a basic cumulative capacity value.

According to another embodiment, the basic cumulative capacity value may be determined based on the number of accesses by the user and an access period. For example, when the first user conducts ten accesses during a unit time, a basic cumulative capacity value for re-determining the mental state of the first user may be determined to the size of setting data, shared data, or the search data generated due to the ten accesses. When the second user conducts fifteen accesses during a unit time, a basic cumulative capacity value for re-determining the mental state of the second user may be determined to the size of setting data, shared data, or the search data generated due to the fifteen accesses.

According to another embodiment, the mental state determiner 110 may be provided in one or more electronic devices to collect basic data, setting data, shared data, search data, or mentality data of one or more users.

According to another embodiment, the mental state determiner 110 may be linked with a mental state determiner provided in another electronic device to provide a function of accessing an online space sharing emotional words selected and input by the user and drawing data created by the user or accessing a networking space forming a connection between users.

The mental state determiner 110 may be driven to access the management server 400 connected via a network.

The management server 400 linked with one or more mental state determiners 110 may receive and manage the basic data, the setting data, the mentality data, the shared data, the search data, and the like of users. The management server 400 may generate a personal space (area or memory) allocated for each user and store the basic data, the setting data, the mentality data, the shared data, the search data, and the like in the personal space.

The management server 400 may generate a space for a group including one or more users, and may store data about activities, actions, histories, and logs within the group. The data about the activities within the group may include a message history or message creation time point between the first user and the second user, a posting created by the first user or a creation time point of the posting, whether the posting is made public, a time point when the posting is made public, a time point when the posting is corrected, whether the posting is corrected, the number of times the posting is corrected, preference information for the posting or creator information of the preference information, a time point when the preference information is created, a time point when the preference information is corrected, and information about a user having a predetermined relationship with the first user (friends, etc.), but embodiments of the present disclosure are not limited thereto. The data about the activities within the group may further include various pieces of information.

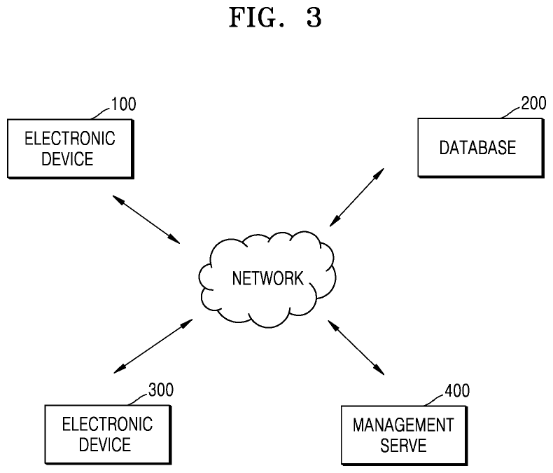

FIG. 3 is a diagram of a mentality determination system according to embodiments of the present disclosure.

The mentality determination system may include the electronic devices 100 and 300 carried by users, the database 200 for managing data for mentality determination, and the management server 400.

The electronic device 100 and/or the electronic device 300 include the mental state determiner 110 and are carried by users, and thus may input basic data about a user, setting data related to a behavior of the user, shared data, search data, and input data input by the user. The basic data about the user may be obtained from the database 200. The basic data about the user may be obtained based on identification information input by the user.

The setting data related to the behavior of the user may be obtained via an input while the mental state determiner 110 is being operated. The setting data related to the behavior of the user may be stored in a memory of the electronic device 100 and then transmitted to and backed up in the database 200. The setting data related to the behavior of the user may be an input with respect to the mental state determiner 110.

The shared data may be created in relation to a sharing action performed in another program provided in the mental state determiner 110 or the electronic device 100. The shared data may be created through an action of opening drawing data, emotional word selection data, and keyword data created by the user, who is a holder of the electronic device, to other users, or may be created in relation to a user action with respect to drawing data, emotional word selection data, and keyword data disclosed by the other users. Illustration of the shared data overlaps with the description given above with reference to FIG. 2, and thus will be omitted.

The search data may be created in relation to a search action performed in the mental state determiner 110. The search data may be created in relation to an action of searching for registered drawing data, registered emotional word selection data, registered keyword data, and the like. Illustration of the search data overlaps with the description given above with reference to FIG. 2, and thus will be omitted.

The electronic device 100 and/or the electronic device 300 may transmit created data to the database 200 or to the management server 400. The management server 400 may transmit received data to the database 200. The management server 400 may process the received data according to a predetermined protocol to generate processed data, and may transmit the processed data to the database 200.

The drawing data, the emotional word selection data, and/or the keyword data obtained by the electronic device 100 may be transmitted to the electronic device 300 carried by a psychological analysis counselor. In this case, the drawing data, the emotional word selection data, and/or the keyword data may be transmitted to the electronic device 300 via the management server 400, or may be transmitted to the electronic device 300 via a path provided by the management server 400.

The electronic device 300 may input psychological analysis data about the received drawing data, the received emotional word selection data, and/or the received keyword data. The psychological analysis data may be transmitted to the electronic device 100 or the management server 400.

When there is a behavior of a user or an input by the user, the mental state determiner 110 stored the electronic device 100 or the electronic device 300 creates data in correspondence with the behavior of the user or the input by the user. The data created in response to the user's behavior or input may include a timestamp value at the time point of the action or input, and an environment information value at the time point of the action or input. Data created in response to the user's action or input may be converted into values by a predefined table.

The management server 400 may receive data from the electronic devices 100 and 300 via a network. The management server 400 may generate a conversion expression of data created in response to the user's action or input, and may distribute the conversion expression to the electronic devices 100 and 300. The management server 400 may manage the degree of relevance to the determination of the mental state for each data and each factor of data. The management server 400 may update the mental state determiner 110 to collect only data or factors whose degree of relevance to the determination of the mental state is equal to or greater than a preset basic determination degree.

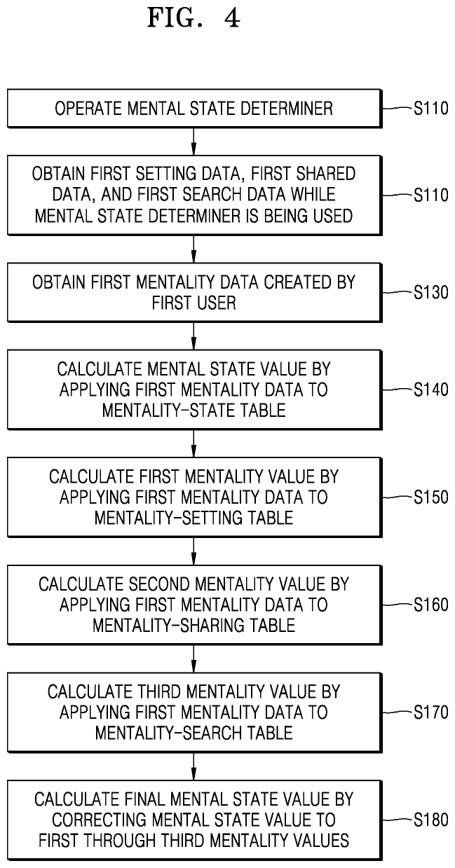

FIG. 4 is a flowchart of a mentality determination method according to embodiments of the present disclosure.

In operation S110, the electronic device 100 executes the mental state determiner 110.

In operation S120, the electronic device 100 may obtain first setting data, first shared data, and first search data while a mental state determiner is being used.

In operation S130, the electronic device 100 may obtain first mentality data created by the first user.

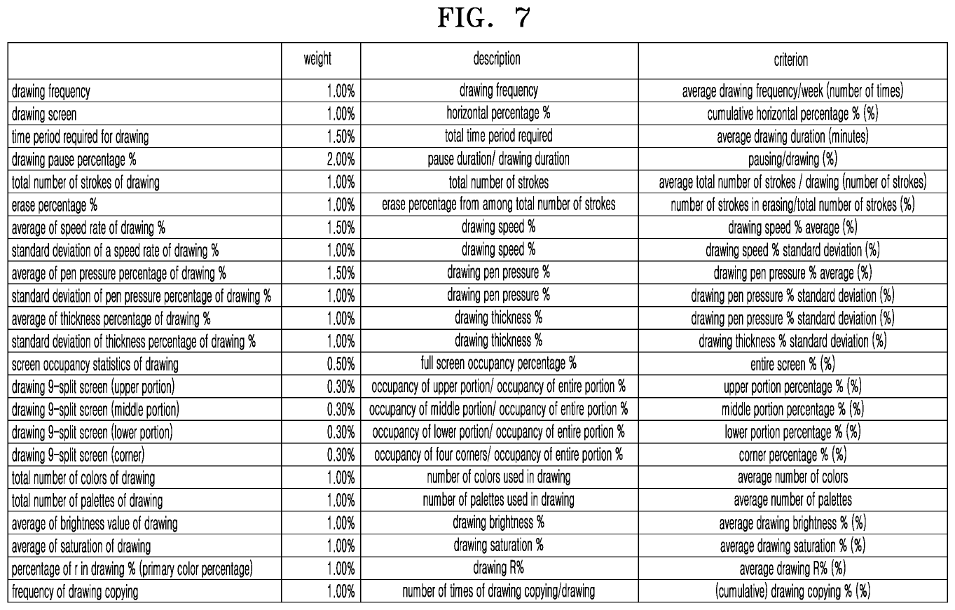

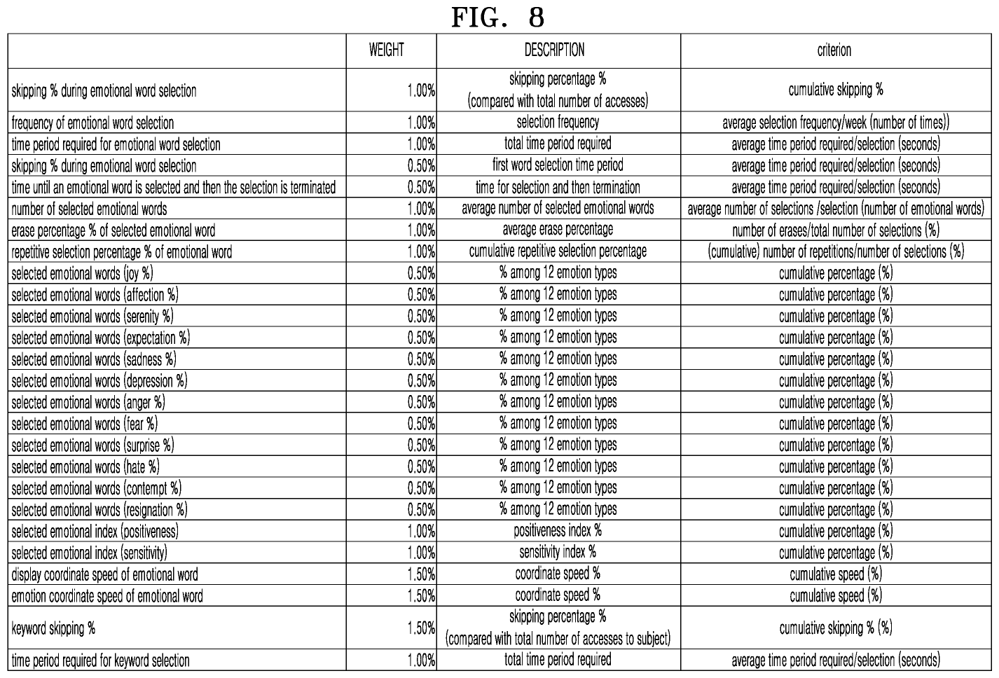

In operation S140, the electronic device 100 may calculate a mental state value by applying the first mentality data to a mentality-state table. The mentality-state table may be at least one of a mentality-drawing table or a mentality-emotional word table. As shown in FIG. 7, the mentality-drawing table may represent a relationship between the factors of drawing data and the type values included in mental states. As shown in FIG. 8, the mentality-emotional word table may represent a relationship between the factors (or setting values) of emotional word data and the type values included in mental states. The electronic device 100 may calculate the mental state value by using this mentality-drawing table and this mentality-emotional word table. The mental state value may be calculated based on type values of one or more mental states, or a value or type value obtained by combining the type values with one another.

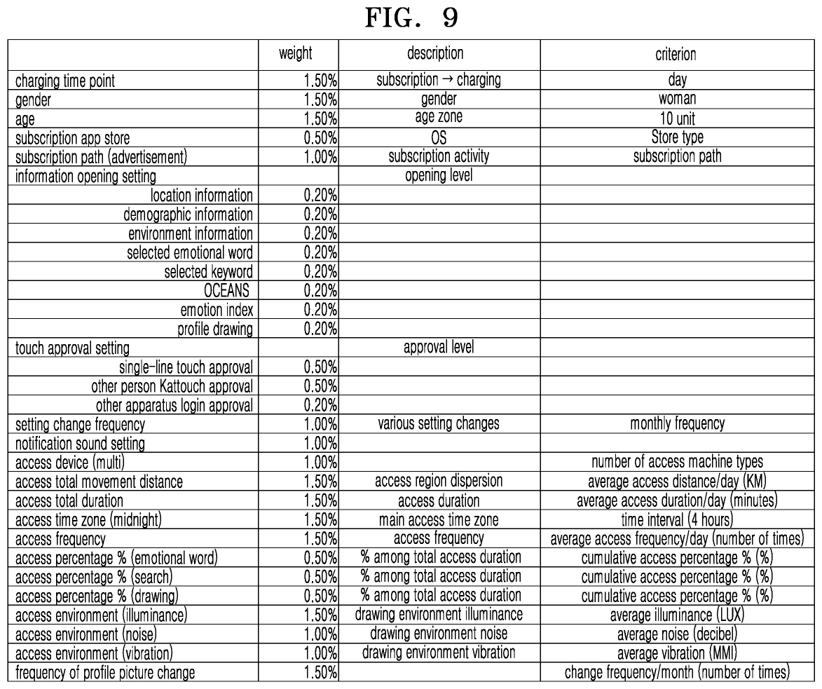

In operation S150, the electronic device 100 may calculate a first mentality value by applying the first mentality data to a mentality-setting table. The electronic device 100 may calculate a first mentality value corresponding to first setting data that is data of a setting input by a user, by applying the first setting data to a mentality-setting table. As shown in FIG. 9, the mentality-setting table may represent a relationship between the factors of setting data and the type values included in mentality values. The first mentality value may be calculated based on type values of one or more mental states, or a value or type value obtained by combining the type values with one another.

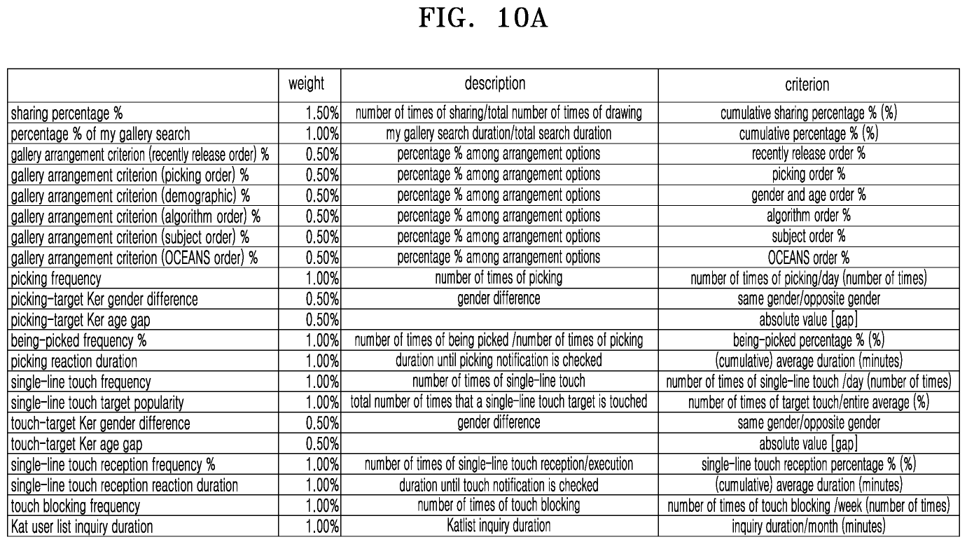

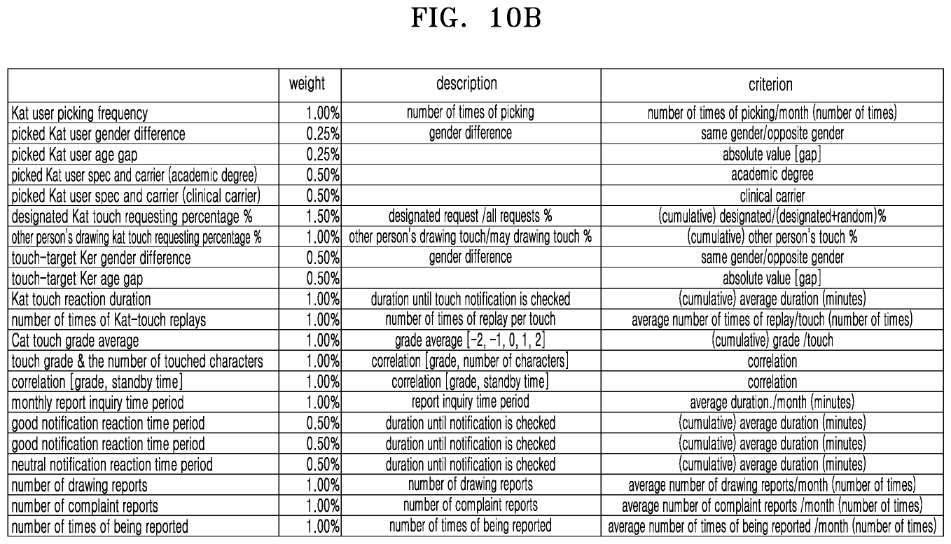

In operation S160, the electronic device 100 may calculate a second mentality value by applying the first mentality data to a mentality-sharing table. The electronic device 100 may calculate a second mentality value corresponding to first sharing data that is data of a sharing input by a user, by applying the first sharing data to a mentality-sharing table. As shown in FIG. 10A, the mentality-sharing table may represent a relationship between the factors of sharing data and the type values included in mentality values. The second mentality value may be calculated based on type values of one or more mental states, or a value or type value obtained by combining the type values with one another.

In operation S170, the electronic device 100 may calculate a third mentality value by applying the first mentality data to a mentality-search table. The electronic device 100 may calculate a third mentality value corresponding to first search data that is data of a search input by a user, by applying the first search data to a mentality-search table. As shown in FIG. 10B, the mentality-search table may represent a relationship between the factors of search data and the type values included in mentality values. The third mentality value may be calculated based on type values of one or more mental states, or a value or type value obtained by combining the type values with one another.

In operation S180, the electronic device 100 may calculate a final mental state value by correcting the mental state value to the first through third mentality values.

The mentality-state table, the mentality-setting table, the mentality-sharing table, and the mentality-search table may be defined differently according to the ages of users. For example, different tables may be applied to users over 14 years of age and users under 14 years of age.

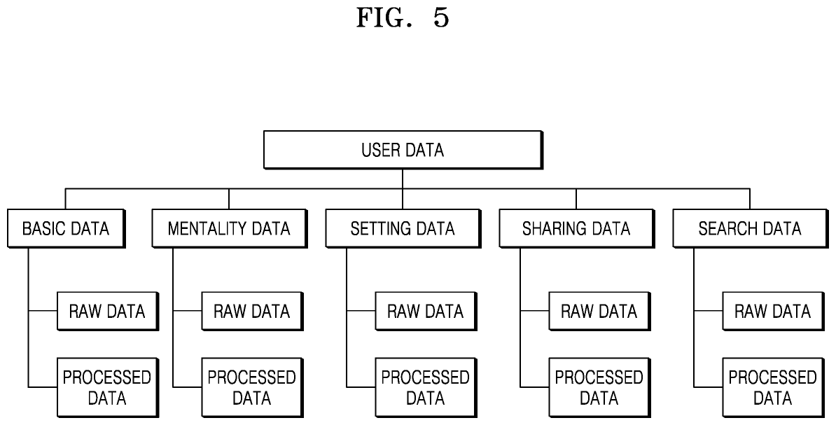

FIG. 5 is a road map of pieces of data obtained by the mental state determiner 110 to determine the mental state of a user.

FIG. 6A is first mentality-factor table representing a correlation between data and a mental state value used in embodiments of the present disclosure. FIG. 6B is second mentality-factor table representing a correlation between data and a mental state value used in embodiments of the present disclosure. FIG. 6C is third mentality-factor table representing a correlation between data and a mental state value used in embodiments of the present disclosure.

The mentality-factor table represents respective factors of mentality data, setting data, basic data, shared data, and search data and type values (openness, conscientiousness, extraversion, agreeableness, neuroticism, and energy) representing mental state values.

As shown in FIG. 6A, the factor of a charging time point is related to openness (O), conscientiousness (C), agreeableness (A), and energy (E), and may have a strong positive relation with openness, a negative relation with conscientiousness, a positive relation with agreeableness, and a positive relation with energy. The earlier the charging time point is, the higher the openness, the agreeableness, and the energy are, and the lower the conscientiousness is. Here the charging time point is when to switch to pay.

As shown in FIG. 6A, examples of the user setting factors used to calculate the mental state are such as a charging time point, a gender, an age, a subscription app, a subscription path (whether subscription through an advertisement is performed), information opening setting, touch approval setting, whether there are multiple connected devices, distances between occurrence locations of connection inputs, an access time zone (morning, afternoon, or evening), an access frequency, a drawing action ratio, a noise value in an access environment, a vibration value in an access environment, the frequency of profile picture change, the number of times (percentage) skipping is selected during emotional word selection, the frequency of emotional word selection, the number of selected emotional words, the percentage of joy in selected emotional words, the percentage of expectation in selected emotional words, the percentage of sadness in selected emotional words, the percentage of depression in selected emotional words, the percentage of anger in selected emotional words, the percentage of fear in selected emotional words, the percentage of surprise in selected emotional words, the percentage of hate in selected emotional words. Also, as shown in the table of FIG. 6A, correlations between the factors and mental state values may be defined. The information opening setting may be whether or not information is disclosed to others. The access total movement distance may be distance moved during access the app.

As shown in FIG. 6B, examples of the user setting factors used to calculate the mental state are the percentage of contempt in selected emotional words, the percentage of resignation in selected emotional words, a change speed of an emotional word display coordinate, a speed of a change between emotional word display coordinates, the frequency of drawing, screen information of drawing (horizontal viewing or vertical viewing), a drawing/erasing total distance percentage, a pen pressure average value of drawing, a pen pressure standard deviation value of drawing, a thickness average value of drawing, a thickness standard deviation value of drawing, screen occupancy statistics (occupancy level) of a drawing, the number of palettes of a drawing, an average value of brightness in a drawing, an average value of saturation in a drawing, percentage information of an R value in a drawing, a criterion of gallery arrangement, OCEANS (mental state values) of Ker user which is to be picked. Also, as shown in the table of FIG. 6B, correlations between the factors and mental state values may be defined. The drawing speed refers to the moving speed of the touch stroke while drawing.

As shown in FIG. 6C, examples of the user setting factors used to calculate the mental state are the frequency of being picked (frequency of a preference action), a picking reaction speed (time until a preference action occurs), OCEANS (mental state values) of picked Ker user, a touch-target Ker demographic gap, touch-target Ker OCEANS, a touch blocking frequency, the number of times of Kat-touch replays, a Kat user touch grade average, a touch grade & the number of touched characters, a touch grade & a touch standby time value, a monthly report inquiry time value, a good notification reaction time period, a bad notification reaction time period, a neutral notification reaction time period, the number of drawing reports, the number of complaint reports, the number of times of being reported, a kids gallery search ratio, a keyword guide inquiry frequency, kids drawing sharing percentage information, sharing percentage information, kids gallery search percentage information, a picking-target kids demographic gap, and picking-target kids OCEANS (mental state values). Also, as shown in the table of FIG. 6C, correlations between the factors and mental state values may be defined. Based on the values, a value corresponding to the energy may be calculated from the mental state values of the user. Ker user or Ker may refer to a user who is a psychological target, and Kat user or Kat may refer to a user who is a psychological analysis counselor. Picking is a user interface provided by a mental state determiner, and may be input of preference information. It can be predefined for the good notifications, bad notifications and neutral notifications. Kids refers to other users associated with each user.

FIG. 7 illustrates the mentality-drawing table used in embodiments of the present disclosure.

The mentality-drawing table may further include weight values for a drawing frequency, the horizontal percentage of a drawing screen, a time period required for drawing, a drawing pause percentage, the total number of strokes of drawing, erase percentage information of drawing, an average value of a speed rate of drawing (speed value of a touch motion), a standard deviation of a speed rate of drawing (standard deviation of a speed value of a touch motion), an average value of the pen pressure percentage of drawing, a standard deviation of the pen pressure percentage of drawing, an average value of the thickness percentage of drawing, a standard deviation of the thickness percentage of drawing, screen occupancy statistics of drawing, a drawing 9-split screen (upper portion) (occupancy degree of an upper portion), a drawing 9-split screen (middle portion) (occupancy degree of a middle portion), a drawing 9-split screen (lower portion) (occupancy degree of a lower portion), a drawing 9-split screen (corner) (occupancy degree of a corner), the total number of colors of a drawing (number of used colors), the total number of palettes of a drawing (number of used palettes), an average value of the brightness value of a drawing (average value of brightness values of pixels), an average value of the saturation of a drawing (average value of saturation values of pixels), the percentage of R in RGB of a drawing (percentage information of the R value of each pixel), the frequency of drawing copying (frequency of usage of a provided drawing copying function), which are included in the drawing data. The weight values may be values corresponding to the degrees to which the factors of the drawing data affect mental state values or respective type values of the mental state value. The weight values may be determined as percentile values, and, when the weight values are summed, the sum is equal to 100%, and thus, a mental state value may be calculated based on the weight values.

FIG. 8 illustrates the mentality-emotional word table used in embodiments of the present disclosure. The weight values for emotional words are defined as shown, and these weight values may be used to mental states.

FIG. 9 illustrates the mentality-setting table used in embodiments of the present disclosure.

The mentality-setting table represents a correlation between setting data and a mental state value. The setting data includes information opening setting and is related to whether information opening has been set. The setting data may include whether location information is open, whether demographic information is open, whether environmental information is open, whether selected sentiment words are open, whether selected keywords are open, whether OCEANS (mental state values) is open, whether an emotional index is open, and whether a profile drawing is open. The emotional index may be an index value calculated from a selected emotional word.

FIG. 10A illustrates a mentality-setting & search table used in embodiments of the present disclosure. FIG. 10B illustrates a mentality-search table used in embodiments of the present disclosure.

In addition to the tables shown in FIGS. 7 through 10A and 10B, various tables between mentality and factors, such as a mentality-basic table and a mentality-keyword table, may be created based on pieces of data of users, and mental state values of the users may be calculated using the created tables.

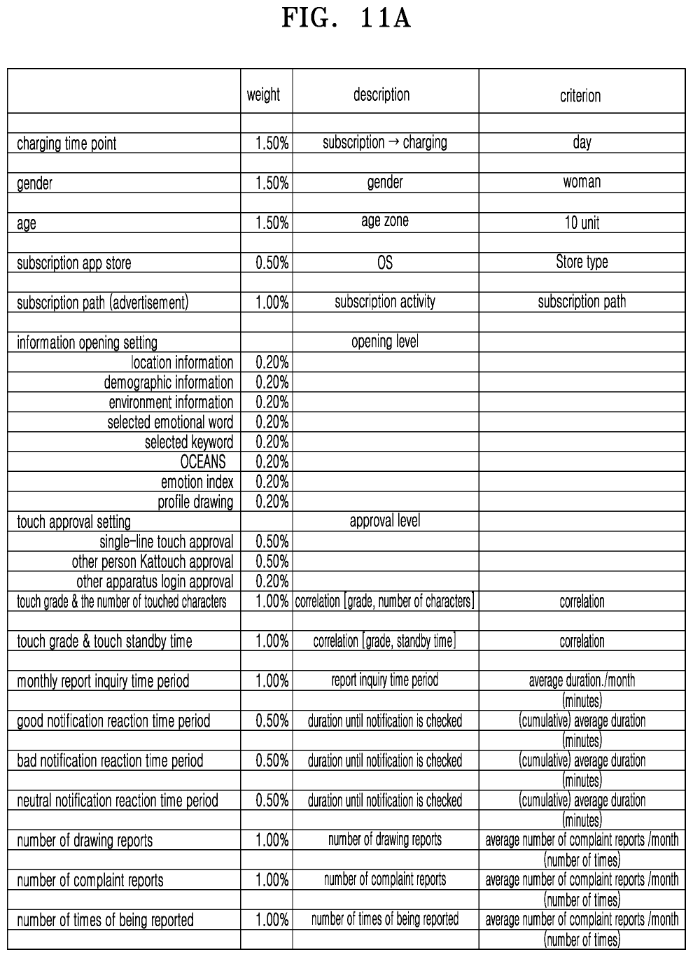

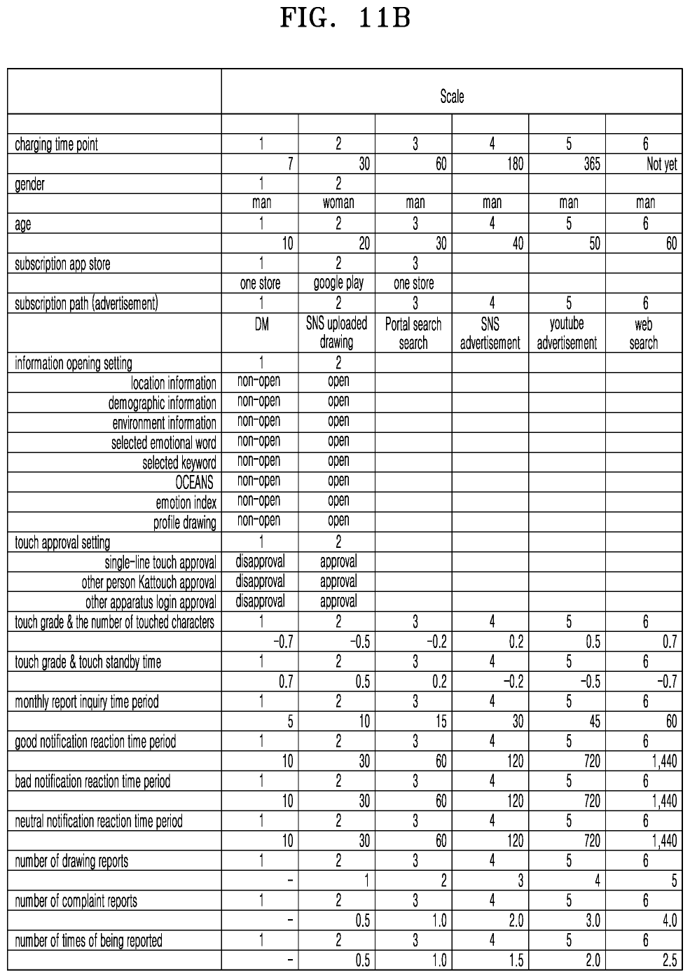

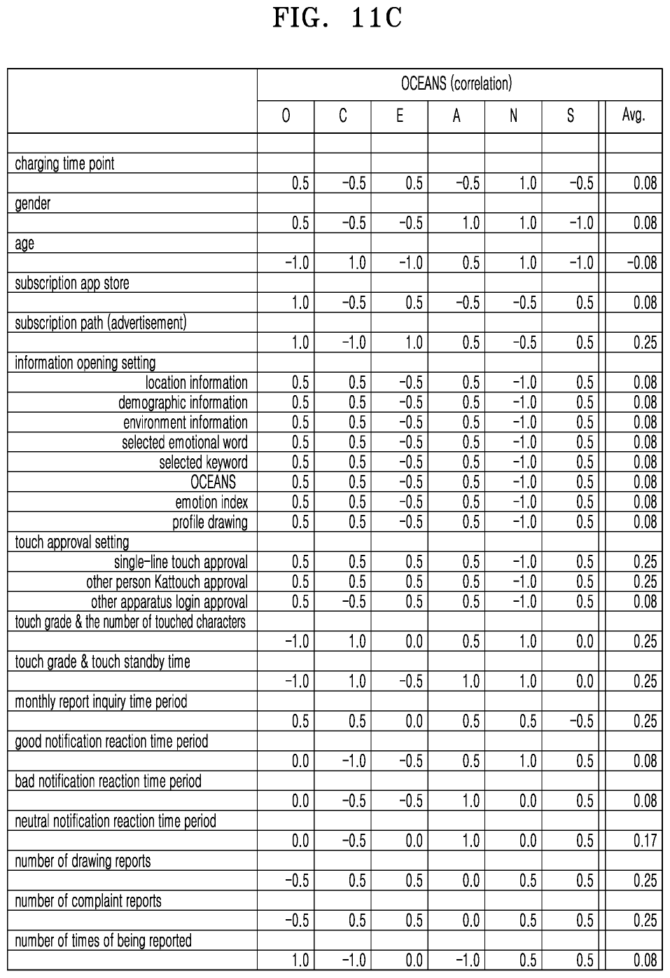

FIG. 11A illustrates a table of description and criterion among a mentality-factor table used in embodiments of the present disclosure; FIG. 11B illustrates a table of magnitude values of each factor among a mentality-factor table; and FIG. 11C illustrates a table of mental state values for factors among a mentality-factor table. The factors are set to one of the predefined scales, and the factors may be defined as correlations with psychological state values.

The electronic device described above may be implemented as a hardware component, a software component, and/or a combination of hardware components and software components. For example, the devices and components described in the embodiments may be implemented using at least one general-use computer or special-purpose computer, such as, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processor may execute an operating system (OS) and one or more software applications running on the OS. In addition, the processor may access, store, manipulate, process, and generate data in response to execution of software. For ease of understanding, a single processor may be described as being used, but one of ordinary skill in the art will recognize that the processor may include a plurality of processing elements and/or a plurality of types of processing elements. For example, the processing device may include a plurality of processors or a single processor, and a controller. The processor may have another processing configuration, such as a parallel processor.

The software may include a computer program, a code, instructions, or a combination of one or more of the foregoing, and may configure the processor so that the processor can operate as intended, or to independently or collectively give instructions to the processing device. The software and/or the data may be permanently or temporarily embodied in any type of machine, component, physical device, virtual equipment, computer storage media or devices, or transmitted signal waves, such that the software and/or the data is interpreted by the processing device or provides an instruction or data to the processing device. The software may be distributed over a networked computer system and stored or executed in a distributed manner. The software and the data may be stored on one or more computer readable recording media.

A method according to an example may be embodied as program commands executable by various computer means and may be recorded on a computer-readable recording medium. The computer-readable recording medium may include program commands, data files, data structures, and the like separately or in combinations. The program commands to be recorded on the computer-readable recording medium may be specially designed and configured for examples or may be well-known to and be usable by one of ordinary skill in the art of computer software. Examples of the computer-readable recording medium include a magnetic medium such as a hard disk, a floppy disk, or a magnetic tape, an optical medium such as a compact disk-read-only memory (CD-ROM) or a digital versatile disk (DVD), a magneto-optical medium such as a floptical disk, and a hardware device specially configured to store and execute program commands such as a ROM, a random-access memory (RAM), or a flash memory. Examples of the program commands are advanced language codes that can be executed by a computer by using an interpreter or the like as well as machine language codes made by a compiler. The hardware devices can be configured to function as one or more software modules so as to perform operations according to examples, or vice versa.

According to an embodiment of the present disclosure, the mental state determiner may estimate and calculate a mental state value of a user, based on cumulatively obtained user's behavior data or input data.

While the disclosure has been particularly shown and described with reference to examples thereof, it will be understood by those of ordinary skill in the art that various changes in form and details may be made therein without departing from the spirit and scope of the disclosure as defined by the following claims. For example, an appropriate result may be attained even when the above-described techniques are performed in a different order from the above-described method, and/or components, such as the above-described system, structure, device, and circuit, are coupled or combined in a different form from the above-described methods or substituted for or replaced by other components or equivalents thereof.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method, performed by an electronic device, of determining a mental state of a user by using behavior data or input data of the user, the method comprising:
operating a mental state determiner;
obtaining first setting data, first shared data, and first search data, based on behavior data or input data according to a behavior of the user who is using the mental state determiner;
obtaining first mentality data created by a first user;
calculating a mental state value by applying the first mentality data to a mentality-state table;
calculating a first mentality value by applying the first setting data to a mentality-setting table;
calculating a second mentality value by applying the first sharing data to a mentality-sharing table;
calculating a third mentality value by applying the first search data to a mentality-search table; and
calculating a final mental state value by correcting the mental state value to the first through third mentality values.

2. The method of claim 1, wherein the first mentality data comprises at least one of an emotional word set input by the user and drawing data input by the user.

3. The method of claim 1, wherein the first setting data comprises at least one of a subscription path, a subscription app, a subscription date and time, a subscription region, a charging time point, profile registration information, an access device, an access date and time, an access duration, an access frequency, an access location, an access proportion, an access environment, and environment setting information, which are related to a subscription behavior of the user.

4. The method of claim 1, wherein the first sharing data comprises whether mentality data created by the user is shared, sharing information, upload percentage information, sharing percentage information to other platforms, direct transmission percentage information of pieces of mentality data, data of preference actions for mentality data of other users, and data of a preference action for the user.

5. The method of claim 1, wherein the first search data comprises at least one of inquiry/reading information for a psychological analysis counselor, inquiry/reading information for a psychological subject, and inquiry/reading information for mentality data.

6. The method of claim 1, wherein the mental state value comprises a value corresponding to openness, conscientiousness, extraversion, agreeableness, neuroticism, and energy related to the mental state of the user.

7. The method of claim 1, wherein the first through third mentality values comprise a value corresponding to openness, conscientiousness, extraversion, agreeableness, neuroticism, and energy related to the mental state of the user.

8. The method of claim 1, wherein at least one of the mentality-state table, the mentality-setting table, the mentality-sharing table, and the mentality-search table represents a correlation between at least one of one or more pieces of mentality data, setting data, shared data, and search data input by the user and a mental state corresponding to the at least one of the one or more pieces of mentality data, setting data, shared data, and search data.

9. The method of claim 1, wherein at least one of the mentality-state table, the mentality-setting table, the mentality-sharing table, and the mentality-search table is created by training based on training data, the training using at least one of pre-created one or more pieces of mentality data, setting data, shared data, and search data as an input and using pre-generated one or more mental states as an output.

10. A non-transitory computer-readable recording medium having recorded thereon a program to be executed by a processor to:
obtain reaction information and profit information for drawing data created by a first user and a second user;
determine the profit information for the drawing data, based on the reaction information for the drawing data; and
determine a profit distribution value of the first user and a profit distribution value of the second user by distributing the profit information in a determined distribution percentage relationship.
